(19) United States
(12) Patent Application Publication
(10) Pub. No.: US 2026/0116850 A1
(43) Pub. Date: Apr. 30, 2026

(54) A PROCESS AND PLANT FOR THE SYNTHESIS OF UREA AND MELAMINE

(71) Applicant: Casale SA, Lugano (CH)

(72) Inventors: Leonardo Marrone, Mercallo (VA) (IT); Simone Gamba, Pagazzano (BG) (IT); Alberto Benedetti, Como (IT)

(73) Assignee: Casale SA, Lugano (CH)

(21) Appl. No.: 19/164,157

(22) PCT Filed: Mar. 8, 2024
(86) PCT No.: PCT/EP2024/056248

§ 371 Date: Sep. 10, 2025

(30) Foreign Application Priority Data
Mar. 10, 2023 (EP) 23161325.8

(51) Int. Cl. C07C 273/12; C07D 251/60

(52) U.S. Cl. CPC C07C 273/12; C07D 251/60

(57) ABSTRACT

An integrated process for the synthesis of urea and melamine, wherein urea synthesized in a urea plant is used to produce melamine in a melamine plant connected to the urea plant, according to a high-pressure non-catalytic melamine synthesis process, wherein melamine is synthesized at a pressure higher than urea synthesis pressure and offgas liberated during the synthesis of melamine are returned, as a gas, to the high-pressure synthesis section of the urea plant.

### FIELD OF THE INVENTION

The invention relates to the field of combined production of urea and melamine.

### PRIOR ART

Urea is produced industrially by reacting NH₃ and CO₂ at high pressure and high temperature. The reaction of ammonia and carbon dioxide produces ammonium carbamate which dehydrates to form urea and water. Due to the thermodynamic equilibrium of the reactions, the effluent of the reaction process is an aqueous solution of urea containing a significant amount of unconverted ammonia and carbon dioxide in the form of ammonium carbamate.

The modern technology for the industrial production of urea is the so-called urea stripping process wherein the reaction effluent is heated in a high-pressure stripper to decompose the ammonium carbamate into gaseous ammonia and carbon dioxide which are then removed from the solution. The vapours extracted from the stripper are condensed in a high-pressure condenser and the so obtained condensate stream is returned to the urea reactor.

The stripper is typically a vertically arranged shell-and-tube apparatus wherein the solution flows in the tubes of an externally heated tube bundle. To facilitate the removal of the gaseous ammonia and carbon dioxide, a stripping medium may be added. For example, the CO2-stripping process uses gaseous CO2 introduced at the bottom of the stripper as a stripping medium. The ammonia-stripping process uses gaseous ammonia whereas the so called self-stripping process uses no added stripping medium.

The urea reactor, the high-pressure stripper and the high-pressure condenser operate substantially at the same pressure and form a so-called urea synthesis loop or high-pressure loop. The loop pressure is typically well above 100 bar, for example around 150 bar or above. The high-pressure loop, particularly in the case of a CO2 stripping plant, may also include a high-pressure scrubber wherein a gas phase removed from the reactor is scrubbed with a recycle carbamate solution from a low-pressure stage.

The urea-containing aqueous solution effluent from the stripper is further processed in one or more recovery sections, for example a low-pressure recovery section or a medium-pressure recovery section followed by a low-pressure recovery section. A recovery section typically includes at least a carbamate decomposer and a condenser where vapours of ammonia and carbon dioxide are condensed to form a recycle solution. The so obtained recycle carbamate solution may be pumped back to the high-pressure loop, e.g. into the high-pressure condenser.

The recovery section produces a purified urea solution, comprising urea, water and unavoidable impurities. This purified urea solution may be used as such or further processed to remove water, for example in an evaporation section, obtaining a highly concentrated solution termed urea melt.

Urea can be produced also with the total-recycle process. Despite being outperformed by the modern stripping plants, this technology is still in use. In a total recycle processes the urea synthesis occurs at high pressure and is then followed by medium-pressure and low-pressure sections where the unreacted carbamate is decomposed and the resulting carbon dioxide and ammonia are recovered. Compared with modern CO2-stripping or ammonia-stripping processes, a total-recycle process lacks a high-pressure decomposition and condensation stage. Ammonia and carbon dioxide are recycled separately in the form of pure liquid ammonia and aqueous carbamate solution. A total recycle process is typically operated with a high N/C molar ratio in the urea synthesis reactor to maximize the conversion.

Melamine can be produced from urea with a low-pressure catalytic process or, preferably, with a high-pressure non-catalytic process. These processes for the synthesis of melamine are familiar to a skilled person. The high-pressure non-catalytic process, which is nowadays preferred, operates at a pressure of 70 bar or above. Basically, urea decomposes to form melamine, ammonia and carbon dioxide. The reaction produces a melamine-containing liquid effluent, called melamine melt, and a gaseous stream which is termed melamine offgas. Said melamine offgas comprise the ammonia and carbon dioxide liberated during the reaction and may contain water vapour and small amounts of melamine.

The integration of a urea plant with a melamine plant is clearly attractive because urea is the starting material for the synthesis of melamine whereas the melamine offgas, being composed predominantly of ammonia and carbon dioxide, can be recycled to the tied-in urea plant. There has been a continuous effort on how to recycle the melamine offgas to the urea plant and many solutions to this task have been proposed.

A first solution is to condense the melamine offgas at a low pressure or medium pressure, usually not greater than 30 bar, to obtain a liquid stream that can be recycled to the urea plant. The melamine offgas may be condensed together with other gaseous streams (typically vapour streams of the urea plant comprising ammonia and CO2) and in the presence of a recycle carbamate solution to promote condensation. This solution is easy to implement but has the noticeable drawback that condensing the melamine gas at such relatively low pressure requires a significant amount of water. Said water is ultimately introduced in the urea synthesis reactor with the recycle stream, which is not desirable because water in the reactor shifts the chemical equilibrium away from formation of urea. Furthermore, the so obtained condensate must be pumped to the much higher urea synthesis pressure, which requires energy. Condensation of the melamine offgas may be performed in a condenser of the urea plant or in a separate offgas condensation section. A separate offgas condensation section may be preferable but represents an additional cost.

To overcome the above drawbacks, it has been proposed to condense the melamine offgas at a high-pressure so that less water is required for condensation and the so obtained solution can be recycled to the urea synthesis section with less energy for pumping. In the prior art, however, the melamine synthesis runs typically at a pressure of 80 to 120 bar whereas the urea synthesis pressure is generally around 140 bar or greater. Even assuming that melamine offgas are released at synthesis pressure, there is still a pressure gap of around 20 bar or more to overcome. This pressure gap is not negligible which means that recycling the offgas condensate still requires a pump which constitutes an expensive item and potentially a source of failure.

WO 98/08808 suggests that melamine offgas is sent directly to any of the items of a urea synthesis loop, such as urea synthesis reactor, high-pressure stripper, high-pressure condenser or scrubber. However, this is only possible if either the melamine synthesis pressure is raised above the urea synthesis pressure, or the urea synthesis pressure is reduced. Reducing the urea synthesis pressure is generally not acceptable; raising the melamine synthesis pressure above 120 bar to meet the urea synthesis pressure typically of at least 140-150 bar has the undesired side effect of increasing the solubility of CO2 in the liquid effluent of the melamine synthesis reactor. The subsequent purification of said melamine-containing liquid stream uses alkaline chemicals and suffers from the increased amount of CO2 in the solution. Particularly, the consumption of alkaline agents may increase, which introduces a cost. This problem is not yet addressed in the prior art.

Another problem to be solved concerns transients wherein the melamine synthesis process is performed at a pressure lower than design pressure and, consequently, a direct recycle of the melamine offgas to the urea synthesis section requires compression or pumping. An example of such transient is startup of the melamine plant while the urea plant is running. In such a condition, an integrated urea-melamine plant designed to recycle offgas directly to the urea synthesis section still requires a pump or a compressor otherwise the reagents contained in the melamine offgas are lost during transients.

An overview of the urea and melamine processes can be found in the literature, for example Meessen, “Urea” and Crews et al. “Melamine and Guanamines”, both in the Ullmann's Encyclopedia of Industrial Chemistry. A process and plant for urea and melamine production wherein the melamine offgas are sent to a condensation section is described in EP 1 716 111.

### SUMMARY OF THE INVENTION

In an integrated urea-melamine plant, the invention aims to provide a novel and more effective solution for recycling the melamine offgas to the urea plant. The invention aims to provide a direct recycling of the melamine offgas to the urea synthesis section without affecting the melamine purification process and equipment. The invention addresses also the problem of how to adapt the melamine offgas recycle to transients such as start-up when the pressure at which melamine is synthesized, and therefore the pressure of the melamine offgas, is lower than the design synthesis pressure.

The above aims are reached with a process according to the claims.

In the invention, melamine is synthesized at a suitably high pressure, so that the melamine offgas can be returned to the urea synthesis section and to the urea reactor without receiving energy from a machine. Related advantages are no need of a pump or compressor for recycling the melamine offgas and no need of a separate offgas condensation section.

An embodiment of the invention concerns synthesis of melamine in a first reaction environment where a melamine melt is produced, followed by a secondary reaction environment where the melamine melt is stripped with gaseous ammonia. An aspect of the invention concerns conditions of the stripping process in the secondary reaction specifically adapted to a higher than usual synthesis pressure of melamine.

A preferred embodiment of the inventive process concerns a transient such as a startup of the melamine plant, wherein the melamine synthesis pressure runs at reduced pressure. During the transient, offgas liberated by the melamine plant is sent temporarily to a medium-pressure recovery section or to a low-pressure recovery section of the urea plant, until the end of the transient. A related advantage is that melamine offgas can be efficiently recycled to the urea plant even during transients.

A further aspect of the invention is an integrated urea-melamine plant according to the claims. The invention may be applied to new plants or revamping of existing plants as well.

### DESCRIPTION OF THE INVENTION

The invention concerns a process for the synthesis of urea and melamine, which is performed in an integrated plant including a urea plant and a melamine plant.

In the invention, urea is synthesized in a urea synthesis reactor operating at a pressure of at least 135 bar. Said urea synthesis reactor is part of a urea synthesis section, which may include additional items such as a stripper or a condenser. For example, in a total-recycle urea plant the urea reactor may be the only item of the urea synthesis section, whereas a urea stripping plant has a synthesis section including, at least, a stripper and a condenser in addition to the urea reactor.

The melamine plant includes a melamine synthesis section working at a melamine synthesis pressure and the melamine offgas is returned to said urea synthesis section of the urea plant in a gaseous form. The melamine offgas is returned to the urea synthesis reactor, in a gaseous form or after condensation, without receiving additional energy from a machine.

The term machine denotes an equipment with moving parts configured to increase energy of a gas or liquid, such as a pump or a compressor. An ejector, which is a static device with no moving parts, is not understood as a machine according to the above definition.

In the invention the melamine offgas, in a gaseous form or after condensation, reach the urea reactor without passing through a machine as above defined. The recycle path of the melamine offgas may include or may not include an ejector according to some embodiments.

In the invention, the melamine synthesis pressure is greater than the working pressure of an item of the urea synthesis loop to which the melamine offgas is recycled (“offgas-receiving item”). Said item may be the urea reactor or another equipment of the urea synthesis section, preferably a high-pressure carbamate condenser (HPCC).

The urea plant includes a urea synthesis section and may include one or more recovery sections. The urea synthesis section includes, at least, a urea synthesis reactor (“urea reactor”). The urea synthesis section may include, in addition to the urea reactor, a stripper and a condenser (also termed “carbamate condenser”). These pieces of equipment operate at a high pressure and therefore the synthesis section is termed “high pressure section”. The term “high pressure” is used because the pressure in the synthesis section is by far greater than the pressure in the downstream recovery section(s). The synthesis section operates typically well above 100 bar, whereas a recovery section may operate at around 15-20 bar (medium-pressure) or less than 5 bar (low-pressure).

The term “synthesis loop” is also used because the unconverted matter contained in the reactor effluent, after stripping and condensation, returns to the reactor.

In a preferred application of the invention, the synthesis loop is isobaric or nearly isobaric.

An isobaric loop is understood as a synthesis loop whose items operate at the same design pressure, therefore all items run at the same pressure apart from small differences due to pressure drops in the connections. Consequently, no machine such as pumps or compressor is installed to maintain circulation within the loop, particularly to feed condensate from the condenser to the reactor. In certain embodiments, an ejector may be provided in an isobaric loop.

A nearly isobaric loop is understood as a loop wherein at least two equipment of the loop operate under a different design pressure of at least 5 bar but however any difference of pressure between items of the loop is not greater than 15 bar. The circulation within the nearly isobaric loop is a natural circulation, possibly boosted by an ejector. The natural circulation may be promoted by putting items at different elevation.

Typically, a CO2-stripping urea plant has an isobaric loop, as above described, whereas an ammonia-stripping urea plant has a nearly isobaric loop wherein the reactor pressure is greater than the pressure in the condenser.

In an interesting embodiment of the invention, the urea synthesis loop includes a high-pressure carbamate condenser (HPCC), a urea reactor and a gravity flow line arranged to send a condensate stream from said condenser to said reactor, wherein said urea synthesis loop does not encompass any ejector on said gravity flow line.

The melamine plant includes a melamine synthesis section and a downstream section for purification of the melamine-containing melt obtained after synthesis. The melamine offgas is withdrawn from the melamine synthesis section.

The melamine synthesis section, in a highly preferred embodiment, includes a primary reaction environment where melamine is produced from a urea feed, followed by a secondary reaction environment wherein a melamine-containing liquid effluent from the primary reactor is stripped with gaseous ammonia. The melamine offgas is withdrawn from said secondary reaction environment. According to a preferred embodiment of the invention, the stripping process in the secondary reaction environment is performed by contacting the melamine-containing liquid with gaseous ammonia in counter-current, the ammonia being dispersed in the liquid and the liquid having a temperature not less than 350° C.

Performing the stripping process in the secondary reaction environment under the above-mentioned conditions helps to remove the extra amount of CO2 dissolved in the melamine-containing liquid, due to the increased pressure.

The melamine offgas does not receive energy from a machine to travel from the melamine plant to the high-pressure urea synthesis loop and finally to the reactor of the urea plant. This means the offgas stream does not go through a compressor or pump to be recycled to the urea reactor.

By performing the melamine synthesis at a pressure above that of the offgas-receiving item, the melamine offgas can be sent to the urea synthesis section in a gaseous state without compression. Additionally, the unconverted reagents (ammonia and CO2) contained in the melamine offgas can ultimately reach the urea reactor, still in the gaseous state or possibly after condensation, without having to increase their pressure with a machine such as a compressor or pump. In some embodiments the melamine offgas, after condensation, may pass through an ejector before reaching the urea reactor.

The melamine offgas is returned to said urea synthesis section of the urea plant in a gaseous form. The offgas may be sent to any item or connection line of the urea synthesis section. Ultimately, the ammonia and carbon dioxide contained in the melamine offgas reach the urea reactor, which may be obtained by sending the melamine offgas directly to the urea reactor, or by sending to said reactor a condensate stream obtained after condensation of the melamine offgas. Said condensate stream may contain the melamine offgas, now in a condensate form, and may further contain a recycle carbamate stream.

In the invention, the melamine offgas is returned to the urea reactor without receiving additional energy from a machine with moving parts, either by compressing the offgas or pumping a liquid stream obtained after condensation of the melamine offgas. This means that, for example, the urea synthesis loop is isobaric having no machine for circulation in the loop, or the urea synthesis loop has natural circulation. Hence the offgas, either before or after condensation, do not pass through a machine to reach the urea reactor.

In a highly preferred embodiment of the invention, as mentioned above, the melamine synthesis section includes a primary reaction environment followed by a secondary reaction environment. Said environments may be in separate pressure vessel, namely a primary reactor and a secondary reactor. Said secondary reactor is also termed stripping reactor or post-reactor. Said environments may also be integrated in the same pressure vessel, for example with the secondary environment arranged coaxially around the first environment. In the following description, reference is made to a primary reactor and a secondary reactor, provided that the two reaction environments may be contained in the same pressure vessel.

In the primary reactor, urea melt reacts under melamine-forming conditions to produce a liquid effluent containing melamine, dissolved CO2 and impurities, which is termed melamine melt. In the secondary reactor, said melamine melt is stripped with gaseous ammonia, essentially to remove the dissolved CO2. The melamine melt may pass from the first reaction environment to the second reaction environment by overflow, when the liquid level in the primary environment has reached a predetermined value. The melamine melt effluent from the secondary reactor is then treated to remove impurities and obtain pure melamine in a downstream purification section.

Performing the melamine synthesis in the above-described primary reactor and secondary reactor (or post-reactor) is known in the field and is described among others in WO 02/100839. A melamine reactor where the secondary environment is integrated coaxially around the primary environment is described in EP 2 918 333. A more recent development of said coaxial melamine reactor is described in WO 2021/123054.

A first stream of melamine offgas is extracted from the primary reactor, comprising ammonia and CO2 produced by the decomposition of urea. A second stream of melamine offgas is removed from the second reactor, comprising CO2 removed from the melamine melt as well as the gaseous ammonia used as a stripping aid. Said two streams can be joined to form the melamine offgas exported from the melamine synthesis section. Said offgas, made predominantly of ammonia and CO2, is preferably washed with the urea melt feed, prior to introduction of the feed into the primary reactor, to remove traces of melamine contained therein. A melamine offgas washing section or melamine offgas scrubber, if provided, is understood to be part of the melamine synthesis section.

In a preferred embodiment of the invention, the stripping process in the secondary reactor is modified to cope with an increased CO2 content in the melamine melt, which is a consequence of the unusually high pressure of melamine synthesis, well above the customary upper limit of around 120 bar.

Said stripping process is performed preferably by contacting the melamine melt with gaseous ammonia in counter-current. Preferably the melamine melt flows downwards and the gaseous ammonia flows upwards.

In a highly preferred embodiment, the invention includes that a liquid level of the melamine melt in the secondary reaction environment is controlled to remain well above all the injection points of the gaseous ammonia.

More in detail, in a preferred embodiment the gaseous ammonia is introduced in the secondary reaction environment by means of one or more inlets which, in operation, are immersed in the melamine melt. Said inlets may be provided by an ammonia gas feeder located at the bottom of the secondary reaction environment. The liquid level of the melamine melt is controlled to remain above all ammonia gas inlets by at least a minimum level, to ensure effective removal of the dissolved CO2. The applicant has found that a preferred level is at least two meters above the ammonia gas inlets.

The level of the melamine melt may be controlled by a suitable liquid level detector whose reading is used to continuously adjust the flow rate of melamine melt withdrawn from the secondary reaction environment.

The melamine synthesis pressure is preferably at least 3 bar greater than the working pressure of the melamine offgas-receiving item of the urea synthesis section, more preferably at least 6 bar greater. In preferred embodiments the melamine synthesis pressure is 3 to 10 bar or 6 to 10 bar greater than the pressure of said item. In an embodiment, said offgas-receiving item may be the urea reactor; consequently, the melamine synthesis pressure may be 3 to 10 bar or 6 to 10 bar greater than the urea reactor pressure.

The urea synthesis pressure in the urea reactor is preferably in the range 135 bar to 150 bar, preferably 135 bar to 145 bar. The melamine synthesis pressure is preferably 140 to 155 bar, provided it is greater than the pressure in the offgas-receiving item. All pressures are given in bar gauge (barg).

In an embodiment, urea is synthesized with a total recycle process at a pressure not greater than 150 bar, for example 144 bar to 148 bar, such as 145 bar. This pressure is significantly lower than the usual pressure in the urea reactor of a total-recycle urea plant, which is typically 180 to 220 bar. Hence, a feature of the present invention is to run a total recycle urea plant at a reactor pressure much lower than conventional, in order to have melamine synthesis at a higher pressure which allows direct recycle of the melamine offgas.

Still according to the invention, the parameters of the reactor of a total-recycle urea plant, including N/C ratio, H/C ratio and inlet temperature, are adjusted to compensate for the lower reaction pressure, particularly to achieve an acceptable conversion of CO2 in the liquid phase. Particularly preferably, the reactor operates with N/C ratio in the range 3.55 to 3.60 such as 3.57, H/C ratio in the range 0.85 to 0.90, such as 0.88, temperature in the range 185 to 190° C., such as 189° C.

In other embodiments, urea is synthesized with a stripping process, preferably a CO2-stripping process. If urea is produced with a stripping process, the urea synthesis loop includes a high-pressure carbamate condenser where the vapours extracted from the stripper are condensed at a high pressure equal or close to reaction pressure. In such a case, the melamine offgas are preferably sent to said high-pressure condenser. Alternatively, the melamine offgas can be sent to the reactor or to the stripper.

In the various embodiments of the invention, a melamine offgas line transports the melamine offgas from the melamine plant to the urea plant. This line may connect for example the offgas scrubber to a destination item of the urea plant. Said destination item may be the urea synthesis reactor or, if provided, a urea stripper or a high-pressure carbamate condenser. In a urea stripping plant, a very preferred location for the introduction of the melamine offgas in the urea process is the vapour line from the high-pressure stripper to the high-pressure condenser. Accordingly, the melamine offgas will be condensed together with the stripper vapours so that ammonia and carbon dioxide contained in the offgas finally return to the urea reactor with the condensate stream produced in said condenser.

The driving force to transport the offgas from the melamine plant to the urea plant and to introduce the offgas in the urea synthesis section is provided entirely by the difference between the melamine synthesis pressure and the urea synthesis pressure. There is no device to increase the pressure of the melamine gas along the melamine gas transport line. In particular, the invention provides that the melamine gas line does not require a machine such as a compressor or a pump.

In some embodiments, an ejector can help the recycle of the condensed melamine off-gas (from the HPPC) to the urea reactor. Such contribution of an ejector however is not essential for the invention and certain embodiments have no ejector on the condensate recycle line from the HPCC to the urea reactor.

The content of CO2 in the melamine melt effluent after stripping in the secondary reaction environment is preferably less than 100 ppm and more preferably less than 50 ppm.

The invention further addresses the issue of startup transients when the melamine synthesis pressure is reduced. The transient may relate for example to a startup or a shutdown. An example is a startup of the melamine plant while the urea plant is running in steady operation. Another example is a shutdown of the urea plant. The urea feed to the melamine plant is immediately cut; however, the melamine plant still produces a residual stream of melamine offgas.

In an embodiment of the invention, the melamine offgas liberated by the melamine plant are sent temporarily to a medium-pressure recovery section or to a low-pressure recovery section of the urea plant, until the end of the transient. In case of a startup, this can be done until the melamine synthesis section reaches full synthesis pressure and the startup phase is completed. After completion of the startup phase the offgas are routed directly to the urea synthesis section.

For example, in certain cases the melamine start up is performed at a selected reduced pressure, such as 90 barg, until the condition of melamine melt overflow from the primary reactor is reached. This may take some hours in a typical melamine plant of industrial scale.

During the transient, the melamine offgas is sent preferably to a medium-pressure condenser or to a low-pressure condenser.

The choice of sending the melamine offgas to a medium-pressure or to a low-pressure section depends on the kind of urea plant. A total recycle urea plant for example includes typically a medium-pressure recovery section followed by a low-pressure recovery section. When a medium-pressure recovery section is present, it is preferred to send the melamine offgas to said section during the transient. Some urea plants do not have a medium-pressure recovery section: this is the case of typical CO2 stripping plants where the high-pressure synthesis section is followed by a low-pressure recovery section. In that case, during transients the melamine offgas are sent to said low-pressure section.

An integrated urea-melamine plant adapted to operate with the above-described process comprises a urea plant and a tied-in melamine plant, and further includes a first offgas line arranged to return the melamine offgas directly to the urea synthesis section during normal operation, and a second offgas line arranged to return the melamine offgas to a medium-pressure recovery section or to a low-pressure recovery section of the urea plant during a transient. The integrated plant includes means configured to return the melamine offgas to the urea plant via the first offgas line during normal operation with the melamine synthesis section at full pressure, and via the second line during a startup transient with the melamine synthesis section running at a lower pressure.

1. A process for the synthesis of urea and melamine, wherein urea synthesized in a urea plant is used to produce melamine in a melamine plant connected to said urea plant, according to a high-pressure non-catalytic melamine synthesis process, and offgas liberated during the synthesis of melamine are returned to the urea plant, wherein:
the urea plant includes a urea synthesis section including a urea synthesis reactor, wherein said reactor operates at a pressure of at least 135 bar;
the melamine plant includes a melamine synthesis section working at a melamine synthesis pressure;
said melamine offgas is returned to said urea synthesis section by sending the melamine offgas to an offgas-receiving item of the urea synthesis section, wherein said melamine synthesis pressure is greater than the working pressure of said offgas-receiving item, and said melamine offgas is returned to said urea synthesis section of the urea plant in a gaseous form and recycled to the urea synthesis reactor directly in a gaseous form or in a liquid stream obtained after condensation, and the melamine offgas is recycled to the urea reactor without receiving additional energy from a machine either by compressing the offgas or pumping said liquid stream obtained after condensation;
said process further including that, during a startup transient wherein the melamine synthesis pressure is smaller than the pressure of said melamine-offgas receiving item, the melamine offgas liberated by the melamine plant are sent temporarily to a medium-pressure recovery section or to a low-pressure recovery section of the urea plant until the end of the transient.

2. The process according to claim 1, wherein:
the melamine synthesis section includes a primary reaction environment where a liquid melamine melt is produced from a urea feed, followed by a secondary reaction environment wherein said melamine melt, effluent from the primary reactor, is stripped with gaseous ammonia, said offgas being withdrawn partly from said primary reaction environment and partly from said secondary reaction environment.

3. The process according to claim 2, wherein the stripping process in the secondary reaction environment is performed by contacting the melamine melt with gaseous ammonia in counter-current, the gaseous ammonia being dispersed in the liquid and the liquid having a temperature not less than 350° C.

4. The process according to claim 2, wherein gaseous ammonia is introduced in the secondary reaction environment by means of one or more ammonia gas inlets immersed in the liquid melamine melt and the level of said melamine melt in the secondary reaction environment is controlled to remain at least at a minimum level above all said inlets of the gaseous ammonia, preferably at least 2 meters above.

5. The process according to claim 1, wherein said offgas-receiving item is the urea synthesis reactor or said offgas-receiving item is a high-pressure carbamate condenser.

6. The process according to claim 1, wherein said urea synthesis reactor operates at a pressure is in the range 135 bar to 150 bar, preferably 135 bar to 145 bar.

7. The process according to claim 1, wherein said melamine synthesis pressure is at least 3 bar greater than the pressure of said offgas-receiving item, preferably at least 6 bar greater.

8. The process according to claim 1, wherein the transport of the offgas from the melamine plant to the urea reactor is not pushed by any ejector.

9. The process according to claim 1, wherein the content of CO2 in the melamine melt effluent after stripping in the secondary reaction environment is less than 100 ppm.

10. The process according to claim 1, wherein, in the melamine plant, the primary reaction environment and the secondary reaction environment are housed in separate pressure vessel or arranged in the same pressure vessel, preferably in a coaxial arrangement around one another.

11. The process according to claim 1, wherein the urea plant is a total recycle urea plant or a stripping urea plant.

12. The process according to claim 1, wherein the urea plant is a total recycle urea plant, the offgas is sent to the urea synthesis reactor which is operated at a pressure of 144 bar to 146 bar, and the reactor operates with N/C ratio in the range 3.55 to 3.60, H/C ratio in the range 0.85 to 0.90, temperature in the range 185 to 190 °C.

13. The process according to claim 1, wherein the urea plant is a stripping plant and the offgas is sent to the high-pressure carbamate condenser of the urea plant and a condensate formed in said condenser, which contains the condensed melamine offgas, is sent to the urea synthesis reactor without passing through an ejector.

14. The process according to claim 1, wherein, during the transient, the offgas is sent to a medium-pressure condenser or to a low-pressure condenser.

15. The process according to claim 1, wherein the urea synthesis section includes high-pressure items arranged in a synthesis loop, said loop being isobaric or wherein the loop include at least two items working at a different design pressure of at least 5 bar and any difference of pressure between items of the loop does not exceed 15 bar.

16. An integrated urea-melamine plant adapted to operate with the process of claim 1, comprising a urea plant and a tied-in melamine plant, the urea-melamine plant including a first offgas line arranged to return the melamine offgas directly to the urea synthesis section during normal operation, and a second offgas line arranged to return the melamine offgas to a medium-pressure recovery section or to a low-pressure recovery section of the urea plant during a transient, the urea-melamine plant including means configured to return the melamine offgas to the urea plant via the first offgas line during normal operation with the melamine synthesis section at full pressure, and via the second line during a startup transient with the melamine synthesis section running at a lower pressure.
